(19) United States
(12) Patent Application Publication
(10) Pub. No.: US 2024/0247050 A1
(43) Pub. Date: Jul. 25, 2024

(54) PROCESS FOR THE PRODUCTION OF RECOMBINANT PROTEINS

(71) Applicant: UCB BIOPHARMA SRL, BRUSSELS (BE)

(72) Inventors: GEOFFREY NORMAN BROWN, SLOUGH, BERKSHIRE (GB); RICHARD BARRY DAVIES, SLOUGH, BERKSHIRE (GB)

(21) Appl. No.: 18/290,024

(22) PCT Filed: May 9, 2022
(86) PCT No.: PCT/EP2022/062469

§ 371 Date: Nov. 9, 2023

(30) Foreign Application Priority Data
May 10, 2021 (GB) 2106627.9

(51) Int. Cl. C07K 16/00

(52) U.S. Cl. CPC C07K 16/00; C07K 2317/14

(57) ABSTRACT

The invention relates to the field of recombinant production of proteins in bacterial host cells. In particular, the invention relates to processes for culturing bacterial host cells for the production of recombinant proteins, wherein the formation of struvite is reduced by keeping the amount of magnesium added during the production phase within particular ranges.

### FIELD OF THE INVENTION

The invention relates to the field of recombinant production of proteins in bacterial host cells. In particular, the invention relates to processes for culturing bacterial host cells for the production of recombinant proteins, wherein the formation of struvite is reduced.

### BACKGROUND OF THE INVENTION

In the field of medicine, the use of biological entities, such as antibodies or antibody-derived molecules, has been constantly gaining presence and importance. With it, the need for controlled manufacturing processes has developed. The commercialization of proteins for medical use requires that they be produced in large amounts, and a lot of effort has been dedicated to improving the culturing of recombinant host cells that express the desired protein and their processing. This has resulted in increased product titres, but as a consequence, higher amounts of biomass/debris and contaminants are also observed at the cell culture level. As the removal of such contaminants may be laborious, it would be preferable to optimise the processes such that formation of undesired contaminants is minimised.

Struvite is a phosphate material with the formula NH₄MgPO₄·6H₂O. Struvite can be formed in the presence of high concentrations of magnesium, ammonium and phosphate. Struvite formation often occurs in wastewater treatment plants (see e.g. Kim et al., 2007). Struvite formation can also occur during bacterial fermentation (Beavon 1962)., Struvite formation can result in formation of undesired precipitates and/or membrane fouling which is problem for the purification of recombinant proteins from host cells, particularly in industrial scale manufacturing of recombinant proteins.

While the use of struvite precipitation for removal of waste or recovery of nutrients from e.g. wastewater has been investigated extensively, very little has been reported on the formation of struvite during the culturing of host cells for recombinant protein production how to prevent or minimise struvite formation while still maintaining good cell growth and protein production.

This is addressed by the present invention.

### SUMMARY OF THE INVENTION

In a first aspect, the invention relates to a process for producing a recombinant protein, comprising the steps of:
a) providing bacterial host cells capable of producing a recombinant protein upon induction,
b) providing an amount of liquid medium,
c) culturing said bacterial host cells in said liquid medium,
d) inducing production of the recombinant protein, and
e) further culturing said bacterial host cells in the liquid medium to produce the recombinant protein,
wherein for the entire process a total amount of between 0.17 g and 0.28 g magnesium per kg of the liquid medium of step (b) is provided, and
wherein said total amount of magnesium is provided stepwise during the culture from the beginning of step (b) to the end of step (e).

In a further aspect, the invention relates to a process for reducing struvite formation, or reducing the risk of struvite formation, during production of a recombinant protein in a bacterial host cell, said process comprising the steps of:
a) providing bacterial host cells capable of producing a recombinant protein upon induction,
b) providing an amount of liquid medium,
c) culturing said bacterial host cells in said liquid medium,
d) inducing production of the recombinant protein, and
e) further culturing said bacterial host cells in the liquid medium to produce the recombinant protein,
wherein for the entire process a total amount of between 0.17 g and 0.28 g magnesium per kg of the liquid medium of step (b) is provided, and
wherein said total amount of magnesium is provided stepwise during the culture from the beginning of step (b) to the end of step (e).

### DETAILED DESCRIPTION OF THE INVENTION

The present invention relates to processes of culturing cells for the production of recombinant proteins. The inventors have surprisingly found that when magnesium is added stepwise to the cell culture medium during the fermentation such that the concentration of magnesium during fermentation is kept below a critical level, there is a significantly reduced risk of formation of struvite precipitates, while good cell growth and recombinant protein production are maintained. Depending on the magnesium concentration in the initial medium, the total amount of magnesium added during the production phase of the culturing process is to be kept within certain ranges to reduce the risks of formation of struvite precipitates while keeping good culture performances. FIG. 1 provides a non-limiting schematic overview of an embodiment according to the invention. Thus, in a first embodiment, the invention relates to a process for producing a recombinant protein, comprising the steps of:
a) providing bacterial host cells capable of producing a recombinant protein upon induction,
b) providing an amount of liquid medium,
c) culturing said bacterial host cells in said liquid medium,
d) inducing production of the recombinant protein, and
e) further culturing said bacterial host cells in the liquid medium to produce the recombinant protein,
wherein for the entire process a total amount of between 0.17 g and 0.28 g magnesium per kg of the liquid medium of step (b) is provided, and
wherein said total amount of magnesium is provided stepwise during the culture from the beginning of step (b) to the end of step (e).

In a second embodiment, the invention relates to a process for reducing struvite formation, or reducing the risk of struvite formation, during production of a recombinant protein in a bacterial host cell, said process comprising the steps of:
a) providing bacterial host cells capable of producing a recombinant protein upon induction,
b) providing an amount of liquid medium,
c) culturing said bacterial host cells in said liquid medium,
d) inducing production of the recombinant protein, and
e) further culturing said bacterial host cells in the liquid medium to produce the recombinant protein,
wherein for the entire process a total amount of between 0.17 g and 0.28 g magnesium per kg of the liquid medium of step (b) is provided, and
wherein said total amount of magnesium is provided stepwise during the culture from the beginning of step (b) to the end of step (e).

In a third embodiment, the invention relates to a process for producing a recombinant protein according to the first and second embodiments, wherein said total amount of between 0.17 g and 0.28 g magnesium is added in three, or four or more steps during the culture from the beginning of step (b) to the end of step (e)

In a fourth embodiment, the invention relates to a process for producing a recombinant protein, comprising the steps of:
a) providing bacterial host cells capable of producing a recombinant protein upon induction,
b) providing an amount of liquid medium,
c) culturing said bacterial host cells in said liquid medium,
d) inducing production of the recombinant protein, and
e) further culturing said bacterial host cells in the liquid medium to produce the recombinant protein,
wherein for the entire process a total amount of between 0.17 g and 0.28 g magnesium per kg of the liquid medium of step (b) is provided, and
wherein out of the total amount of magnesium provided a first amount of magnesium is in the liquid medium of step (b), a second amount of magnesium is added as a supplement during step (c) and a third amount of magnesium is added as a supplement during step (e).

In a fifth embodiment, the invention relates to a process for reducing struvite formation, or reducing the risk of struvite formation, during production of a recombinant protein in a bacterial host cell, said process comprising the steps of:
a) providing bacterial host cells capable of producing a recombinant protein upon induction,
b) providing an amount of liquid medium,
c) culturing said bacterial host cells in said liquid medium,
d) inducing production of the recombinant protein, and
e) further culturing said bacterial host cells in the liquid medium to produce the recombinant protein,
wherein for the entire process a total amount of between 0.17 g and 0.28 g magnesium per kg of the liquid medium of step (b) is provided, and
wherein out of the total amount of magnesium provided a first amount of magnesium is in the liquid medium of step (b), a second amount of magnesium is added as a supplement during step (c) and a third amount of magnesium is added as a supplement during step (e).

In sixth embodiment, the total amount of magnesium provided for the process according to any of the first to fifth embodiments is between 0.18 g and 0.27 g per kg of liquid medium of step (b), or between 0.19 g and 0.26 g per kg of liquid medium of step (b). In further embodiments, the total amount of magnesium provided for the process according to any of the embodiment of the invention is about 0.17, about 0.18, about 0.19, about 0.20, about 0.21, about 0.22, about 0.23, about 0.24, about 0.25, about 0.26 and about 0.27 g per kg of liquid medium of step (b) (as well as any intermediated values thereof).

In a seventh embodiment, step (c) in the process according to any of the first to sixth embodiments comprises addition of a second amount of magnesium between 0.04 and 0.22 g per kg of the liquid medium of step (b). As a further non-limiting embodiments, step (c) comprises addition of a second amount of magnesium between 0.06 and 0.20 g per kg of the liquid medium of step (b). As an additional non-limiting embodiment, step (c) comprises addition of a secondary amount of magnesium between 0.08 and 0.18 g per kg of the liquid medium of step (b). In other non-limiting embodiments, step (c) comprises addition of a second amount of magnesium of about 0.06, about 0.07, about 0.08, about 0.09, about 0.10, about 0.11, about 0.12, about 0.13, about 0.14, about 0.15, about 0.16, about 0.17 or 0.18 g per kg of the liquid medium of step (b) (as well as any intermediated values thereof). In any of the embodiments of the process according to the invention, magnesium added as a secondary amount during step (c) may e.g. be added as a bolus amount (i.e. a single dose added at one time) and/or be added as part of the feed of a fed-batch phase.

In one embodiment in the process according to any of the embodiments described herein, step (c) consists of a batch phase followed by a fed-batch phase, wherein a bolus of magnesium is added during the batch phase and additional magnesium is added during the fed-batch phase.

In an eighth embodiment, the total amount of ammonium provided for the process according to any of the first to seventh embodiments is at least 2 g of ammonium per kg of liquid medium of step (b), but preferably not more than 20 g, e.g. from 4 to 20 g, from 5 to 15 g or from 6 to 12 g of ammonium.

In a ninth embodiment, the total amount of phosphate provided for the process according to any of the first to eighth embodiments is at least 1 g of phosphate per kg of liquid medium of step (b), but preferably not more than 20 g, e.g. 3 to 15 g, for example 5 to 12 g or 5 to 10 g of phosphate. The bacterial host cells of step (a) of the process according to any of the first to eighth embodiments are capable of producing a recombinant protein upon induction. The bacterial host cells being used in the context of the invention as a whole can be any appropriate bacterial host cell which is suitable for the recombinant production of proteins and able to grow under the specified conditions. In a preferred embodiment, the bacterial host cell is an E. coli cell or a Bacillus cell. In a more preferred embodiment, the host cell is an E. coli host cell, e.g. an E. coli host cell of strain K12, HB101, B7, RV308, DH1, HMS174, W3110 or BL21 or an E. coli strain that is protease deficient. Typically, a nucleic acid sequence encoding the recombinant protein under the control of an inducible promoter has been introduced into the bacterial host cell. Suitable vectors for expressing such nucleic acid constructs in host cells and processes for transformation or transfection of host cells are well-known in the art. Suitable inducible promoters are also well-known in the art and some, non-limiting, examples are mentioned herein below.

In step (c) of the process according to any of the first to ninth embodiments said bacterial host cells are cultured in a liquid medium. Methods and media for the culturing of various types of bacterial host cells are well-known in the art. Media vary according to the organism, but typically comprise components such as a carbon source, a nitrogen source, a phosphorus source, essential metal ions and possibly trace elements (minimal media). They may further comprise additional components such as amino acids and vitamins (rich media) (see e.g. Elbing et al., 2019). Step (c) of the process according to any of embodiments of the invention typically includes fed-batch culturing in a bioreactor. The fed-batch phase may be preceded by a batch phase. Inoculation may occur directly from a working cell bank or via a seed culture, e.g. in a shake flask. A main objective of step (c) of the process according to any of embodiments of the invention is to obtain sufficient biomass for the subsequent protein production phase. In one embodiment, step (c) comprises growing the culture to an OD600 (Optical density at a wavelength of 600 nm) of at least 20, such as at least 25, at least 35, at least 50, at least 55, at least 60, at least 70, or at least 80, preferably to an OD600 of between 20-80 and more preferably between 20-55 or 25-50.

Addition of magnesium to bacterial host cell cultures generally promotes growth. Magnesium has many roles in cells, including involvement in stabilization of membrane phospholipids, lipopolysaccharides, polyphosphate compounds like DNA and RNA, and the ribosome.

Magnesium is also required to make ATP biologically active and participates in catalysis of certain enzymatic reactions through either direct or indirect mechanisms. In the context of the present invention, sufficient magnesium should be added for optimal growth and viability, but magnesium levels should not exceed certain threshold concentrations in order to avoid or minimise struvite formation. Therefore, according to the invention, magnesium needs to be added stepwise during the fermentation process. Magnesium is typically added in the form of a magnesium salt.

In a tenth embodiment, the invention relates to a process for producing a recombinant protein, comprising the steps of:
a) providing bacterial host cells capable of producing a recombinant protein upon induction,
b) providing an amount of liquid medium,
c) culturing said bacterial host cells in said liquid medium,
   i) adding a bolus of magnesium when the OD600 is between 20 and 55,
   ii) further culturing said bacterial host cells whereby the OD increases until the dissolved oxygen (DO) increases to ≥50% of air saturation,
   iii) adding further magnesium and cultivating the host cells until the OD600 increases by at least 15, 20, 25, 35, 40 or 50 units, preferably between 15-50 and more preferably between 20-40 units above the OD600 mentioned in step (c)(i),
d) inducing production of the recombinant protein, and
e) further culturing said bacterial host cells in the liquid medium to produce the recombinant protein,
wherein for the entire process a total amount of between 0.17 g and 0.28 g magnesium per kg of the liquid medium of step (b) is provided, and
wherein said total amount of magnesium is provided stepwise during the culture from the beginning of step (b) to the end of step (e).

In an eleventh embodiment, the invention relates to a process for reducing struvite formation, or reducing the risk of struvite formation, during production of a recombinant protein in a bacterial host cell, said process comprising the steps of:
a) providing bacterial host cells capable of producing a recombinant protein upon induction,
b) providing an amount of liquid medium,
c) culturing said bacterial host cells in said liquid medium to an OD600,
   i) adding a bolus of magnesium when the OD600 is between 20 and 55,
   ii) further culturing said bacterial host cells whereby the OD increases until the dissolved oxygen (DO) increases to ≥50% of air saturation,
   iii) adding further magnesium and cultivating the host cells until the OD600 increases by at least 15, 20, 25, 35, 40 or 50 units, preferably between 15-50 and more preferably between 20-40 units above the OD600 mentioned in step (c)(i),
d) inducing production of the recombinant protein, and
e) further culturing said bacterial host cells in the liquid medium to produce the recombinant protein,
wherein for the entire process a total amount of between 0.17 g and 0.28 g magnesium per kg of the liquid medium of step (b) is provided, and
wherein said total amount of magnesium is provided stepwise during the culture from the beginning of step (b) to the end of step (e).

It is well known that the increase of DO is linked to exhaustion of the carbon source.

As a non-limiting embodiment, magnesium can be added as a bolus in step (c)(i) of the process in an amount between 0.03 g and 0.12 g per kg of the liquid medium of step (b) and the total amount of magnesium added as a supplement in step (c)(iii) may correspond to between 0.01 g and 0.10 g of magnesium per kg of the liquid medium of step (b). As a further non-limiting embodiment, magnesium can be added as a bolus in step (c)(i) in an amount between 0.04 g and 0.11 g per kg of the liquid medium of step (b) and the total amount of magnesium added as a supplement in step (c)(iii) may correspond to between 0.02 g and 0.09 g of magnesium per kg of the liquid medium of step (b). As an additional non-limiting embodiment, magnesium can be added as a bolus in step (c)(i) in an amount between 0.05 g and 0.10 g per kg of the liquid medium of step (b) and the total amount of magnesium added as a supplement in step (c)(iii) may correspond to between 0.02 g and 0.08 g of magnesium per kg of the liquid medium of step (b). In some non-limiting embodiments, magnesium can be added as a bolus in step (c)(i) at about 0.03, about 0.04, about 0.05, about 0.06, about 0.07, about 0.08, about 0.09, about 0.10, about 0.11 or about 0.12 g magnesium per kg of the liquid medium of step (b) (as well as any intermediated values thereof) and the total amount of magnesium added as a supplement in step (c)(iii) may correspond to about 0.01, 0.02, 0.03, about 0.04, about 0.05, about 0.06, about 0.07, about 0.08, about 0.09, about 0.10 of magnesium per kg of the liquid medium of step (b) (as well as any intermediated values thereof).

In another embodiment, the magnesium added as a supplement in step (c)(iii) is added as part of the main feed (i.e. a feed comprising the source of carbon). Alternatively, the magnesium may be added as a supplementary feed, added concurrently or separately from the main feed. Said feed can be an intermittent feed or a continuous (uninterrupted) feed.

In one embodiment, step (d) is initiated when the DO increases to 50% of air saturation in the culture or when a predefined OD600 is reached as defined in step (c)(iii). DO may be measured by any standard means such as an online polarographic dissolved oxygen sensor, an optical dissolved oxygen sensor or any other appropriate oxygen sensing technology.

In step (d) of the process, the production of the recombinant protein is induced. In one embodiment, there is no production, or no significant production, of the recombinant protein prior to step (d). Induction of production of the recombinant protein can be achieved by any suitable method. In one embodiment, a gene encoding the recombinant protein is under the control of an inducible promoter. A number of such inducible promoters is known in the art. A well-known bacterial expression system using an inducible promoter, is a system wherein the gene encoding the recombinant protein is placed under the control of a lac-type promoter, which can be induced by IPTG (Isopropyl β-D-I-thiogalactopyranoside). Other known bacterial expression systems include e.g. the araBAD promoter system (see e.g. Guzman et al., 1995) or the T7/lac system (see e.g. Rosenberg et al., 1987). These and other systems have e.g. been reviewed in Rosano and Ceccarelli (2014).

In an embodiment of the process of the invention, the bacterial host cells comprise a nucleic acid sequence encoding the recombinant protein under the control of an IPTG-inducible promoter and thus produce recombinant protein upon induction with IPTG. In such an embodiment, step (d) of the process comprises addition of IPTG. Alternatively, the bacterial host cells may e.g. comprise a nucleic acid sequence encoding the recombinant protein under the control of an arabinose-inducible promoter (e.g. araBAD promoter), a tryptophan-inducible promoter (e.g. trp promoter) or a phosphate-inducible promoter (e.g. a phoA promoter) and thus produce recombinant protein upon induction with arabinose, tryptophan or phosphate, respectively. In such embodiments, step c) comprises addition of arabinose, tryptophan or phosphate.

In step (e) of the process of the invention the bacterial host cells are being cultured in order to produce the recombinant protein.

Step (e) typically includes fed-batch culturing in a bioreactor. In one embodiment, the duration of step (e) of the process according to any of the embodiments of the invention is between about 10 and about 96 hours, such as between about 12 and about 72 hours, e.g. between about 15 and about 55 hours, such as between 1 about 8 and about 50 hours.

In a non-limiting embodiment of the process according to any of the embodiments of the invention, the total amount of magnesium added as a supplement during step (e) of the process is between 0.02 g and 0.08 g per kg of the liquid medium of step (b). In a further non-limiting embodiment of the process according to any of the embodiments of the invention, the total amount of magnesium added as a supplement during step (e) is between 0.02 g and 0.07 g per kg of the liquid medium of step (b). In an additional non-limiting embodiment, the total amount of magnesium added as a supplement during step (e) is between 0.03 and 0.06 per kg of the liquid medium of step (b). In some specific non-limiting examples, the total amount of magnesium added as a supplement during step (e) of the process according to any of the embodiments of the invention is about 0.02, about 0.03, about 0.04, about 0.05, about 0.06, about 0.07 or about 0.08 g per kg of the liquid medium of step (b) (as well as any intermediated values thereof).

According to the invention, in order to reduce or prevent struvite precipitation, the total amount of magnesium provided for the process is between 0.17 g and 0.28 g per kg of the liquid medium of step (b). Should the liquid medium be depleted or very low in magnesium, the total amount of magnesium to be added during the process [i.e. steps (c) and (e)] may be between 0.17 g and 0.28 g per kg of the liquid medium of step (b). As most liquid media commonly used comprise magnesium, the total amount of magnesium to be added in steps (c) and (e) will have to be adapted, as it will depend on the content of magnesium in the liquid medium. Recipes for liquid media are well-known. For instance, the skilled person would know that a M9 minimal medium comprises about 0.002 g/kg of magnesium whereas the Durany medium comprises about 0.01 g/kg of magnesium. As a non-limiting embodiment, should the total amount of magnesium be targeted at 0.20 g/kg of the liquid medium of step (b), and starting from a M9 minimal medium the skilled person could add a total of 0.198 g of magnesium/kg of the liquid medium during steps (c) and (e) for example by adding a bolus of 0.1 g/kg, an exponential feed providing 0.05 g/kg and a production phase feed providing 0.048 g/kg. In a further non-limiting embodiment, should the total amount of magnesium be targeted at 0.18 g/kg of the liquid medium, and starting from a Durany minimal medium the skilled person could add a total of 0.17 g of magnesium/kg of the liquid medium during steps (c) and (e) for example by adding a bolus of 0.1 g/kg, an exponential feed providing 0.025 g/kg and a production phase feed providing 0.045 g/kg.

In a further embodiment, a feed containing a carbon source (herein also called “main feed”) is added starting step (c)(iii) of the process of the invention. Preferably, the amount of carbon source added to the culture per time unit is lower in step (e) than in step (c)(iii), by lowering the feed rate or by reducing the concentration of the carbon source in the feed.

As described herein, the process of the invention includes culturing bacterial host cells in the presence of a source of magnesium, such as magnesium salts.

As mentioned, magnesium plays a key role in the growth and metabolic functions of microbial and animal cells, and Mg²⁺ availability in cell culture and fermentation media can dramatically influence growth and metabolism of cells.

The magnesium salt provided in the invention may consist of a mixture of magnesium salts or a single magnesium salt. Any magnesium salt suitable for the growth or microbial or animal cells may be used, including, but not limited to, magnesium sulphate, magnesium chloride or yet magnesium boride, as such or under any of their hydrate forms. In one embodiment, the magnesium salt provided does not include significant amounts of magnesium phosphate. Preferably, the magnesium salt does not comprise any salt of magnesium and phosphate. In a preferred embodiment, the magnesium salt comprises or consists of magnesium sulphate or magnesium chloride, as such or under any of their hydrate forms.

The liquid medium can be a minimal medium or a rich medium, such as (but not being limited to) M9, M63, Durany, LB, TNT or derivative media therefrom (see e.g. Elbing et al., 2019).

The liquid medium typically comprises magnesium. In one embodiment, the liquid medium of step (b) of the process of the invention comprises a total amount of magnesium between 0.001 g and 0.25 g of magnesium per kg of liquid medium, e.g. from 0.01 g to 0.25 g, from 0.02 to 0.25 g, from 0.02 to 0.2 g, 0.02 to 0.1, such as (about) 0.02, 0.03, 0.04, 0.05, 0.06, 0.07, 0.08 or 0.09 g magnesium per kg of the liquid medium of step (b) of the process according to any of the embodiments of the invention.

The liquid medium typically comprises also ammonium. Ammonium salts are important sources of nitrogen. Any ammonium salt or mixture of ammonium salts suitable for the growth or microbial or animal cells may be used, including, but not limited to ammonium sulphate, ammonium phosphate, ammonium chloride and ammonium carbonate. Preferably, the ammonium salt does not comprise any salt of ammonium and phosphate. In one embodiment, the total amount of ammonium provided for the process is at least about 2 g of ammonium per kg of the liquid medium of step (b) of the process of the invention, but preferably not more than 20 g, e.g. from 4 to 20 g, from 5 to 15 g or from 6 to 12 g, such as (about) 6.0, 6.5, 7, 7.5, 8, 8.5, 9, 9.5, 10, 10.5, 11, 11.5 or 12 g of ammonium per kg of the liquid medium of step (b).

The liquid medium of step (b) typically also comprises phosphate. In one embodiment, the liquid medium comprises phosphate corresponding to a total of at least 1 g of phosphate per kg of the liquid medium of step (b) but preferably not more than 20 g/kg, e.g. 3 to 15 g/kg or 5 to 12 g/kg, such as about 5, 6, 7, 8, 9, 10, 11 or 12 g of phosphate per kg.

The process of the invention typically includes the addition of one or more organic carbon sources. The carbon source used may be a single type of carbon source or a mixture of different carbon sources. Suitable carbon sources include e.g. glucose, lactose, arabinose, glycerol, sorbitol, galactose, xylose or mannose. As an example, more than 75%, e.g. at least 90%, of the carbon source in the culture medium in step b) consists of glycerol. In another preferred embodiment, more than 75%, e.g. at least 90%, of the carbon source in the culture medium in step (e) of the process of the invention consists of glycerol. As another example, more than 75%, e.g. at least 90%, of the carbon source in the culture medium in step (c) consists of glucose. In another preferred embodiment, more than 75%, e.g. at least 90%, of the carbon source in the culture medium in step (e) consists of glucose. As a further example, more than 75%, e.g. at least 90%, of the carbon source in the culture medium in step (c) consists of lactose. In another preferred embodiment, more than 75%, e.g. at least 90%, of the carbon source in the culture medium in step (e) consists of lactose.

The formation of struvite has been described to be influenced by pH (see e.g. Pérez-García et al., 1989). In an embodiment of the process of the invention, the pH of the culture in step (c) of the process according to any of the embodiments of the invention is above 6.5, such as 6.6, 6.7, 6.8, 6.9, 7.0, 7.1, 7.2 and the pH of the culture in step (e) is above 6.5, such as 6.6, 6.7, 6.8, 6.9, 7.0, 7.1, 7.2. In another embodiment, the pH in step (c) is between 6 and 8, such as between 6.5 and 7.5, e.g. between 6.6 and 7.4, such as between 6.7 and 7.3, e.g. between 6.8 and 7.2 and the pH in step (e) is between 6 and 8, such as between 6.5 and 7.5, e.g. between 6.6 and 7.4, such as between 6.7 and 7.3, e.g. between 6.8 and 7.2. The temperature is typically kept as constant as possible throughout the full fermentation process.

The recombinant protein produced in the process of the invention is typically a heterologous protein, originating from another organism. For example, the recombinant protein may be an antibody, cytokine, growth factor, hormone or other peptide or polypeptide.

In a preferred embodiment, the recombinant protein is an antibody. The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG₁, IgG₂ₐ, IgG_{2b}, IgG₃, IgG₄, IgE, IgD and antibodies that are produced as dimers of this basic structure including IgGA₁, IgGA₂, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe) or Fab-(dsscFv)₂ (also referred to as TrYbe, see WO2015/197772 for instance). Antibody fragments as defined above are known in the art. In a preferred embodiment, the recombinant protein produced is a Fab or a Fab′ fragment.

The process according to any of the embodiments of the invention can in principle take place in any suitable container such as a shake flask or a bioreactor, which may or may not be operated in a fed-batch mode depending e.g. on the scale of production required. In a preferred embodiment, at least steps (c), (d) and (e) are performed in a bioreactor, preferably an industrial scale bioreactor. The bioreactor may e.g. be a stirred-tank or air-lift reactor. The bioreactor may be a reusable reactor made of glass or metal, e.g. stainless steel, or a single-use bioreactor made of synthetic material, such as plastic. In a preferred embodiment, at least step (e) of the process of the invention is carried out in a bioreactor with a volume of equal or more than 100 L, equal or more than 500 L, equal or more than 1,000 L, equal or more than 2,000 L, equal or more than 5,000 L, equal or more than 10,000 L or equal or more than 20,000 L, 1,000 to 30,000 L, 5,000 to 30,000 L, 10,000 to 30,000 L, 1,000 to 20,000 L, 5,000 to 20,000 L, 10,000 to 20,000 L or 10,000 to 25,000 L. In a preferred embodiment, in step (b), (c), (d) or (e) the culture has a volume of equal or more than 100 L, equal or more than 500 L, equal or more than 1,000 L, equal or more than 2,000 L, equal or more than 5,000 L, equal or more than 10,000 L or equal or more than 20,000 L, 1,000 to 30,000 L, 5,000 to 30,000 L, 10,000 to 30,000 L, 1,000 to 20,000 L, 5,000 to 20,000 L, 10,000 to 20,000 L or 10,000 to 25,000 L. In a further preferred embodiment, in all of steps (b), (c), (d) and (e) the culture has a volume of equal or more than 100 L, equal or more than 500 L, equal or more than 1,000 L, equal or more than 2,000 L, equal or more than 5,000 L, equal or more than 10,000 L or equal or more than 20,000 L, 1,000 to 30,000 L, 5,000 to 30,000 L, 10,000 to 30,000 L, 1,000 to 20,000 L, 5,000 to 20,000 L, 10,000 to 20,000 L or 10,000 to 25,000 L.

The process of the invention may comprise one or more further steps after step (e). For instance, the process may comprise the further step of recovering the recombinant protein, which may comprise first separating cells from supernatant or from inclusion bodies. Once recovered, the recombinant protein can be isolated and purified. Isolation and purification processes are well-known to those skilled in the art. They typically consist of a combination of various chromatographic and filtration steps. The process of the invention may further comprise the step of formulating the recombinant protein into a pharmaceutical composition suitable for medical use, e.g. therapeutic or prophylactic use. In one embodiment, the recombinant protein is modified, such as conjugated to another molecule, before being formulated into a pharmaceutical composition.

### DESCRIPTION OF THE FIGURES

FIG. 1: Overview of the phases of a process according to the invention: During the batch phase the cells are grown on the bathed carbon source. Once a defined OD600 is reached a magnesium bolus is added. When the batched carbon source is depleted the feed is started (Exp. Feed) and the cells continue to grow. The batch and Exp. Feed phases correspond to step (c) as described herein. Then after induction of expression of the heterologous protein [Inducer added-corresponding to step (d)] the cells continue to be cultivated while heterologous protein is produced [corresponding to step (e)].

FIG. 2: FIG. 2a is a pellet taken from a fermentation bioreactor where struvite was generated (struvite can be seen as the white spot indicated by the arrow) whilst FIG. 2b is from a fermentation with no struvite present.

FIG. 3: Magnesium concentration in supernatant samples taken during the fermentation process from experiments carried out with differing levels of magnesium in the bolus (grams of magnesium per kg of liquid medium of step (b)).

FIG. 4: Magnesium concentration in supernatant samples taken during the fermentation process described during Example 4.

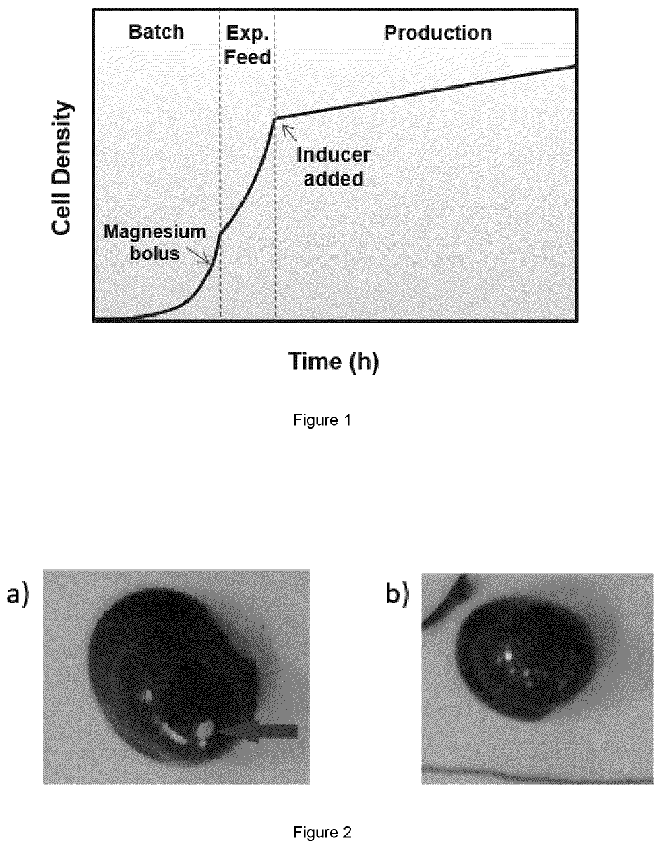

### EXAMPLES

### Material and Methods

In the following Examples, the process is performed as follows unless otherwise stated:

Culture of the cells: A frozen cell bank vial containing E. coli W3110 host cells expressing Antibody A (a Fab′ fragment having a pl in the range 8.8-9.3.) was used to inoculate a shake flask (700 mL total volume) containing 6× peptone-yeast extract (6×P-Y) medium plus tetracycline. This shake flask was incubated at 30° C. and 200-250 rpm. At the required OD range, the shake flask was used to inoculate a seed fermenter (20 L total volume) containing a chemical defined medium (derived from the MD media from Durany et al. 2004, and containing about 0.05 g/kg of Mg) plus tetracycline, with a carbon source. The cell culture within the seed fermenter was maintained at 30° C., dissolved oxygen concentration (DO) was maintained above 20% of air saturation and pH was controlled at about 7.0. At the required OD range, the seed culture was used to inoculate the production fermenter (175 L liquid medium) containing the same chemically defined medium as used in the seed fermenter. The production fermenter was maintained in the same conditions as the seed fermenter and grown in the batch phase until the carbon source was depleted. During this time a bolus addition of MgSO₄ was made to avoid depletion of this metabolite (see all the Examples). At the end of the batch phase (signalled by a spike in the measured DO), an exponential carbon source feed (containing magnesium at a level of about 0.06 g/kg liquid medium of step (b) was switched on and the culture was fed with a specific amount of carbon source to achieve an OD600 of greater than 50 units. At this point, the carbon source feed was switched from an exponential phase feed to a Production phase feed (see all the Examples) and the Antibody A expression was induced by the addition of IPTG. Cells (containing the expressed Antibody A) were harvested after more than 40 hours post induction.

Struvite analysis: For each sampling point, triplicates of 1 mL broth culture were centrifuged in 2 ml Eppendorf tubes, supernatants were discarded, and cell pellets dried by inversion of tubes according to standard methods. Pellets were further dried in an oven at 110° C. for ≥24 h, then visually inspected to qualitatively assess the presence of struvite by comparison to reference images of pellets with struvite (as shown in FIG. 2a) and without struvite (as shown in FIG. 2b).

Magnesium analysis: Concentration of magnesium in samples was determined according to manufacturer's instructions using Quantichrom Magnesium Assay kit (BioAssay Systems, cat #DIMG-250) and a FLUOstar OPTIMA micro plate reader (BMG LABTECH).

DO measurement: Dissolve oxygen (DO) was measured using an online polarographic dissolved oxygen sensor.

### Example 1: Process A

The fermentation was carried out as described above. Samples were taken during the fermentation and analysed for magnesium levels as well as for the presence of struvite. The amounts of magnesium added via the bolus and Production phase feed are showing in Table below:

| | g/kg of liquid |
|---|---|
| | medium of step (b) |
| Total magnesium in bolus | 0.1 |
| Total magnesium in | 0.09 |
| production phase feed | |

In this example, the total amount of magnesium provided to the cells all along the fermentation process (via the liquid medium, bolus, exponential feed and linear feed) was above 0.3 g per kg of liquid medium of step (b).

Struvite was clearly visible in the pellets resulting from samples taken at the harvest point (data not shown) and as such it can be concluded that the bolus and feed concentrations used are not suitable. Because of the drawbacks of struvite precipitations in bioreactors, the process needed to be amended. As the molar quantity of magnesium provided for the process was at least five times lower than that of each one of phosphate and ammonium, it was a hypothesis from the inventors that struvite precipitation was most significantly influenced by the total amount of magnesium provided for the fermentation process (i.e. magnesium added as a bolus, in the exponential feed addition and in the production phase feed, in addition to the amount of magnesium comprised in the liquid medium).

### Example 2: Effect of Reducing Mg in the Production Phase Feed and Varying Mg Bolus Amount

As the inventors did not intend to modify the composition of the liquid medium nor of the exponential feed, they decided to study the effect of magnesium added as a bolus and in the production phase feed. The fermentations were carried out as described in the material and methods section. Samples were taken during the fermentation and analysed for magnesium levels as well as for the presence of struvite. The amount of magnesium in the Production phase feed was set at about 0.04 g/kg liquid medium of step (b), for all three conditions. This is approximately half as much as used in the Production phase feed described in Example 1. The level of magnesium added in the bolus was varied as shown in the table below.

| | Magnesium in bolus |
|---|---|
| | per kg liquid medium |
| Condition | of step (b) (g/kg) |
| 1 | 0.06 |
| 2 | 0.08 |
| 3 | 0.1 |

In this example, the total amount of magnesium provided to the cells all along the fermentation process (via the liquid medium, bolus, exponential feed and linear feed) was between about 0.2 and 0.25 g per kg of the liquid medium of step (b).

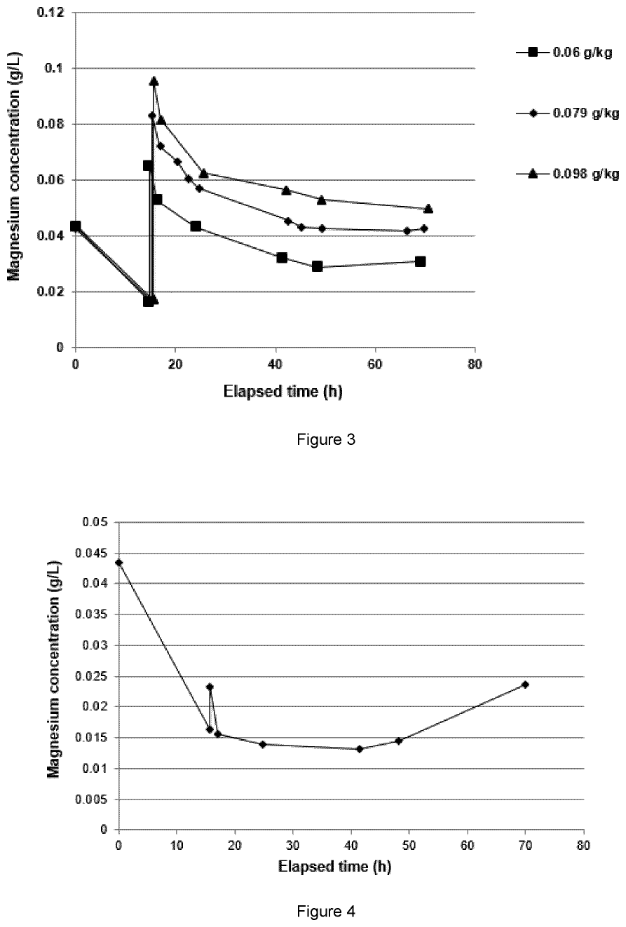

FIG. 3 shows that the adaptations to the amounts used in the Production phase feed and bolus had the expected impact on the resulting magnesium level in the fermentation broth.

Visual inspection of the pellets resulting from samples taken at the harvest point clearly shows that none of the assessed conditions resulted in the formation of struvite. Further, cell growth was not impacted (data not shown).

### Example 3: Impact of Further Bolus Reduction

The fermentation was carried out as described in the material and methods section. Samples were taken during the fermentation and analysed for magnesium levels as well as for the presence of struvite. The concentrations of magnesium added in the bolus and feed are showing in Table below:

| | g/kg of liquid |
|---|---|
| | medium of step (b) |
| Magnesium in bolus | 0.01 |
| Total magnesium in | 0.05 |
| production phase feed | |

The magnesium amount added in the bolus in this Example is an 83% reduction compared to the mid-point of the study reported in Example 2. The total amount of magnesium provided to the cells all along the fermentation process was below about 0.16 g per kg of the liquid medium of step (b). Visual inspection of the pellets resulting from samples taken at the harvest point clearly shows that these concentrations did not result in the formation of struvite (data not shown). FIG. 4 shows that the magnesium concentration fell to a low level and remained low for a considerable time, which had a negative impact on the growth of the cells, leading to the later increase in magnesium level (as the reduction in growth resulted in the cells not utilising as much of the magnesium that was being added via the feed).

### Overall Conclusion:

It was a surprising finding of the inventors that struvite precipitations can be reduced or avoided, while maintain good culture performance, by keeping the total amount of magnesium provided for the culture between about 0.17 and about 0.28 g/kg of the liquid medium of step (b) and adding the magnesium stepwise.

### REFERENCES

Beavon & Heatley (1962) J. Gen. Microbiol.; 31:167-169
Kim et al. (2007) Appl Microbiol Biotechnol 75: 187
Baneyx (1999) Curr Opin Biotechnol 10: 411
Guzman et al. (1995) J Bacteriol 177: 4121
Rosenberg et al. (1987) Gene 56:125
Rosano and Ceccarelli (2014) Front Microbiol 5:172
Elbing, K. L., & Brent, R. (2019) Current Protocols in Molecular Biology, 125, e83.
Durany et al. (2004). Process BioChem 39:1677-1684
Pérez-García et al. (1989) Chemosphere 18:1633

1.-16. (canceled)

17. A process for producing a recombinant protein, comprising the steps of:
(a) providing bacterial host cells capable of producing a recombinant protein upon induction,
(b) providing an amount of liquid medium,
(c) culturing said bacterial host cells in said liquid medium,
(d) inducing production of the recombinant protein, and
(e) further culturing said bacterial host cells in the liquid medium to produce the recombinant protein,
wherein for the entire process a total amount of between 0.17 g and 0.28 g magnesium per kg of the liquid medium of step (b) is provided, and
wherein said total amount of magnesium is provided stepwise during the culture from the beginning of step (b) to the end of step (e).

18. The process according to claim 17, wherein out of the total amount of magnesium provided a first amount of magnesium is in the liquid medium of step (b), a second amount of magnesium is added as a supplement during step (c) and a third amount of magnesium is added as a supplement during step (e).

19. The process according to claim 18, wherein the total amount of magnesium added as a supplement during step (e) is between 0.02 g and 0.08 g per kg of the liquid medium of step (b).

20. The process according to claim 18, wherein the magnesium supplement added during step (c) comprises addition of a total amount of between 0.04 g and 0.22 g magnesium per kg of the liquid medium of step (b).

21. The process according to claim 17, wherein step (c) comprises growing the culture to an OD600 of at least 35.

22. The process according to claim 17, wherein step (d) is initiated when the dissolved oxygen increases to ≥50% of air saturation in the culture.

23. The process according to claim 17, wherein step (c) comprises the following steps:
i) culturing said bacterial host cells,
ii) adding a magnesium supplement corresponding to between 0.03 g and 0.12 g of magnesium per kg of the liquid medium of step (b) when the OD600 is between 20 and 55,
iii) culturing said bacterial host cells until the dissolved oxygen increases to ≥50% of air saturation,
iv) adding a total amount of between 0.01 g and 0.10 g magnesium per kg of liquid medium of step (b) and culturing the host cells until the OD600 of the culture has increased by at least 20 units above the OD600 mentioned in step (ii).

24. The process according to claim 17, wherein the magnesium is added in the form of a salt.

25. The process according to claim 24, wherein the magnesium salt is magnesium sulphate or magnesium chloride.

26. The process according to claim 17, wherein said bacterial host cells are E. coli cells.

27. The process according to claim 17, wherein the pH of the culture in step (c) is between 6 and 8 and/or wherein the pH of the culture in step (e) is between 6 and 8.

28. The process according to claim 17, wherein the duration of step (e) is between 10 and 96 hours.

29. The process according to claim 17, wherein the process comprises the step of recovering the recombinant protein.

30. The process according to claim 29, wherein the process further comprises the step of purifying the recombinant protein and, optionally, a further step of formulating the recombinant protein.

31. The process according to claim 17, the process being an industrial scale process.

32. The process according to claim 17, wherein step (e) is carried out in a bioreactor with a volume of equal or more than 100 L.

33. A process for reducing struvite formation, or reducing the risk of struvite formation, during production of a recombinant protein in a bacterial host cell, said process comprising the steps of:
(a) providing bacterial host cells capable of producing a recombinant protein upon induction,
(b) providing an amount of liquid medium,
(c) culturing said bacterial host cells in said liquid medium,
(d) inducing production of the recombinant protein, and
(e) further culturing said bacterial host cells in the liquid medium to produce the recombinant protein,
wherein for the entire process a total amount of between 0.17 g and 0.28 g magnesium per kg of the liquid medium of step (b) is provided, and
wherein said total amount of magnesium is provided stepwise during the culture from the beginning of step (b) to the end of step (e).
